(19) United States
(12) Patent Application Publication
(10) Pub. No.: US 2006/0149202 A1
(43) Pub. Date: Jul. 6, 2006

(54) Line of sanitary napkins with proportional wings

(76) Inventors: Jeannine Cardin, St-Eugene (CA); Helmut Georgi, Drummondville (CA); Eve-Marie Roy, Drummondville (CA)

Correspondence Address: ST. ONGE STEWARD JOHNSTON & REENS, LLC, 986 BEDFORD STREET, STAMFORD, CT, 06905-5619, US

(21) Appl. No.: 11/295,046

(22) Filed: Dec. 6, 2005

Related U.S. Application Data
(60) Provisional application No. 60/632,983, filed on Dec. 6, 2004.

(51) Int. Cl. A61F 13/15

(52) U.S. Cl. 604/385.04; 604/385.02

(57) ABSTRACT

A line of feminine hygiene products comprising a plurality of sanitary napkins of different lengths (Ln) tailored to meet different menstrual flow needs, each sanitary napkin being provided with a pair of lateral wings of a given length (Lw), wherein the Lw/Ln ratio is substantially constant for all the sanitary napkins of the line. The product line of the present invention advantageously adapts the length of the wings to the particular needs of the wearer having selected a napkin of a given length. The comfort of the wearer is therefore optimized, while minimizing “bunching” effects when unnecessary.

### PRIOR APPLICATION

Applicants claim priority benefits under 35 U.S.C. §119(e) of U.S. Provisional Patent Application Ser. No. 60/632,983 filed Dec. 6, 2004.

### FIELD OF THE INVENTION

The present invention relates to the field of sanitary napkins and more particularly concerns a product line of such napkins.

### BACKGROUND OF THE INVENTION

It is well known in the art to provide sanitary napkins with foldable wings. The ratio between the length of the wing and the length of the napkin is a factor affecting both the comfort of the user and the lateral protection against side leakage.

To meet specific needs of the users, sanitary napkins are usually offered as part of a line of products having similar characteristics but various sizes, lengths, shapes, thicknesses, scents or levels of absorbance. Among the known product lines, there are the ones sold under trademarks such as Kotex or Always. For example PROCTER & GAMBLE, which brand is Always, owns the three following patents or patent applications presenting product lines: U.S. Pat. No. 6,601,705 (MOLINA et al.); CA 2,459,199 (STULTS et al.) and U.S. Pat. No. 6,604,609 (BRUCE et al.). MOLINA et al. discloses a line of sanitary undergarments having a different degree of absorbance, STULTS et al. presents a line also including tampons and BRUCE et al. provides a line of undergarments shaped in accordance with the crotch section of an underwear.

In either case, the wings, when present, are commonly all of the same size, irrespectively of the length of a given napkin. This factor can be a source of discomfort for the wearer or can affect the side protection by being too short. Other product lines such as those sold under the trademark Stayfree do provide wings of different lengths, but there is no clear correlation between the length of the napkin and the length of its wings.

On one hand, long wings are known to create unnecessary strain in the wearer's undergarments, which may be particularly unacceptable in the case of shorter napkins used by wearers wishing only a minimal protection. This issue has been addressed in some products by providing folds or extensibility on either side of the wings, but this solution renders more complex the production of such napkins and increases production costs. On the other hand, long or “night-time” products with shorter wings may provide inadequate coverage, leading to side leakage.

The following documents present examples of prior art sanitary napkins with wings:

CA 2,459,199
EP 1 269 952
US 2003/0093054
US 2003/0195488
US 2004/0068244
US 2004/0133179
US 2004/0138636
US 2004/0236298
US 2004/0243087
US 2004/0260263
U.S. Pat. No. 4,687,478
U.S. Pat. No. 4,790,838
U.S. Pat. No. 4,940,462
U.S. Pat. No. 5,154,715
U.S. Pat. No. 5,221,275
U.S. Pat. No. 5,389,094
U.S. Pat. No. 5,507,735
U.S. Pat. No. 5,558,663
U.S. Pat. No. 5,584,829
U.S. Pat. No. 5,672,165
U.S. Pat. No. 5,704,930
U.S. Pat. No. 5,827,258
U.S. Pat. No. 5,853,401
U.S. Pat. No. 5,961,508
U.S. Pat. No. 6,328,722
U.S. Pat. No. 6,395,956
U.S. Pat. No. 6,416,502
U.S. Pat. No. 6,527,757
U.S. Pat. No. 6,601,705
U.S. Pat. No. 6,604,609
U.S. Pat. No. 6,616,644
U.S. Pat. No. 6,632,208
U.S. Pat. No. 6,716,206

For examples of prior art documents disclosing pleated wings, see U.S. Pat. No. 6,328,722 or U.S. Pat. No. 4,790,838.

There is therefore a need for a product line where the size of the wings on each napkin is better adapted to the napkin's intended use.

### SUMMARY OF THE INVENTION

An object of the present invention is to provide a line of feminine hygiene products that satisfies the above-mentioned need.

In accordance with the invention, that object is achieved with a line of feminine hygiene products comprising a plurality of sanitary napkins of different lengths (Ln) tailored to meet different menstrual flow needs, each sanitary napkin being provided with a pair of lateral wings of a given length (Lw), wherein the Lw/Ln ratio is substantially constant for all the sanitary napkins of the line.

In other words, the present invention provides a line of feminine hygiene products including a plurality of sanitary napkins of different lengths, each provided with a pair of lateral wings having a length, and a proportional relationship between the length of the lateral wings and the length of the corresponding napkin being substantially the same for all the napkins of the product line.

Preferably, the length of the wings corresponds to approximately 32 to 34% of the length of the corresponding napkin. Also preferably, the wings are provided with a step on either side thereof improving protection along the edges of the napkin.

The product line of the present invention advantageously adapts the length of the wings to the particular needs of the wearer having selected a napkin of a given length. The comfort of the wearer is therefore optimized, while minimizing “bunching” effects when unnecessary.

Further features and advantages of the present invention will be better understood upon reading of preferred embodiments thereof with reference to the appended drawings.

### BRIEF DESCRIPTION OF THE DRAWINGS

FIGS. 1A to 1D are top views of four different sanitary napkins of a product line according to a preferred embodiment of the invention.

FIGS. 2A to 2E are top views of five different sanitary napkins of a product line according to another preferred embodiment of the invention.

### DESCRIPTION OF PREFERRED EMBODIMENTS OF THE INVENTION

In the present description, it will be understood that the expression “sanitary napkins” is used to designate any sanitary product adapted to be positioned in the crotch section of an undergarment irrespectively of its construction, level of absorbency or shape, including panty liners. Typically, sanitary napkins have a napkin body comprising a top sheet; a back sheet and an absorbent core disposed therebetween (see for example several of the prior art documents mentioned above in the prior art section). As used herein, the term “wings” refers to any type of flaps extending laterally outward from the napkin body (see for example, the prior art documents mentioned in the above prior art section). The construction of a sanitary napkin with flaps is readily apparent to one of ordinary skill in the art, particularly in light of the many currently marketed sanitary napkins with wings, and the documents mentioned in the prior art section, and as such does not need further description.

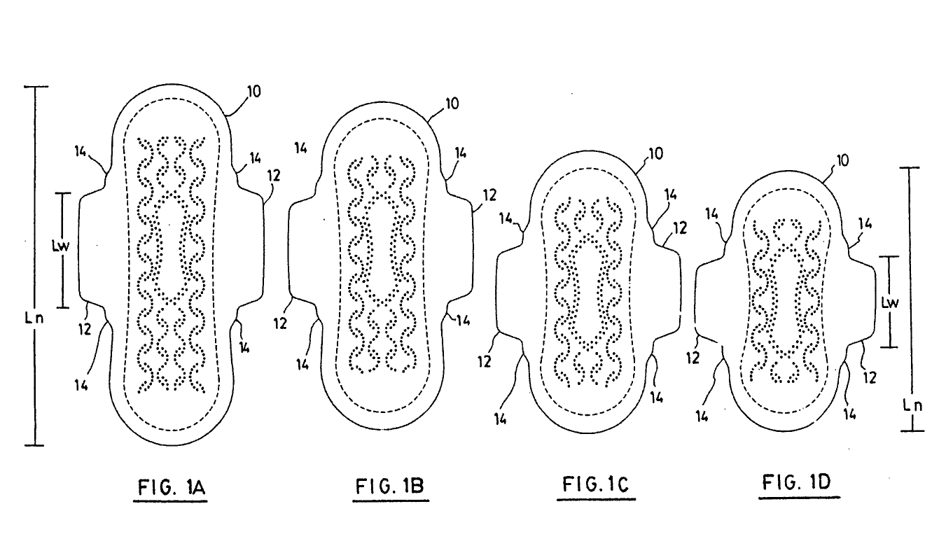

The present invention provides a line of sanitary products including a plurality of sanitary napkins of different lengths, that is, two or more, tailored to meet different flow needs. Each sanitary napkin is provided with lateral wings, the proportional relationship between the length of the lateral wings and the length of the corresponding napkin being the same for all the napkins of the product line. Referring to FIGS. 1A to 1D, there are shown exemplary napkins 10 with pairs of lateral wings 12 of a product line according to a preferred embodiment of the invention. The napkins 10 of this product line are all of the “Ultrathin” construction, and all have similar shapes and absorbency characteristics. The lengths of the illustrated products are respectively labelled “night-time”, “long”, “regular” and “slender”, but it will be understood that these choices and designations are given as examples only and are in no way limitative to the present invention.

More specifically, the line of sanitary products according to the invention preferably comprises one sanitary napkin of a first length (see FIG. 1D) tailored to meet a light menstrual flow need. This napkin of a first length is also referred to as “slender”, and is preferably 220 mm long. The line further comprises a sanitary napkin of a second length (see FIG. 1C) tailored to meet a medium menstrual flow need. The napkin of the second length is also referred to as a “regular”, and is preferably approximately 240 mm long.

A third length is provided within the line which is tailored to meet a heavy menstrual flow need (see FIG. 1B). This napkin is also referred to as “long” sanitary napkin. The length of the long sanitary napkin is approximately 280 mm.

The line may also further comprise a sanitary napkin of a fourth length (see FIG. 1A) tailored to meet a super heavy menstrual flow need. This fourth length is also referred to as the “night time” length and it is approximately 310 mm long.

As can be appreciated, the product line of the present invention advantageously adapts the length of the wings to the particular needs of the wearer having selected a napkin of a given length. Thanks to the fact that the proportional relationship between the wings and its corresponding napkin is always the same for all the sanitary napkins of the line, the comfort of the wearer is optimized, while minimizing bunching effects. There is therefore no need to provide the sanitary napkins with pleated wings. The comfort of the wearer is simply obtained by adjusting the length of the wings with the length of the napkin.

The product line of the present invention may include any appropriate number of “winged” models according to the present invention, and may further include wingless models without departing from the scope of the invention.

For all the illustrated napkins 10, the ratio between the length Lw of the wings 12 and the total length Ln of the napkin 10 is the same. Preferably, the value of the Lw/Ln ratio is between 32 and 34% (or 0.32 and 0.34). As for example, the length Lw of the wings of the slender napkin shown in FIG. 1D is 70 mm and the length of the napkin is 220 mm, whereas the length of the wings of the regular napkin shown in FIG. 1C is approximately 80 mm, and the length of the napkin is approximately 240 mm, and so on with the napkins illustrated in FIGS. 1A and 1B.

It will be understood however that the invention is not limited to such values and that other proportion ratios could be considered without departing from the scope of the present invention. It will also be noted that the position of the wing 12 along the length of the napkin 10 can vary from one size to the next, and is preferably selected to optimize the comfort of the wearer.

In accordance with another feature of the invention, the wings 12 on each napkin 10 are preferably provided with steps 14 on either side thereof, forming a shoulder at the junction of the corresponding wing and the side edge of the napkin it is attached to. The presence of these steps 14 provides a better protection for the wearer's undergarments without creating uncomfortable strains therein.

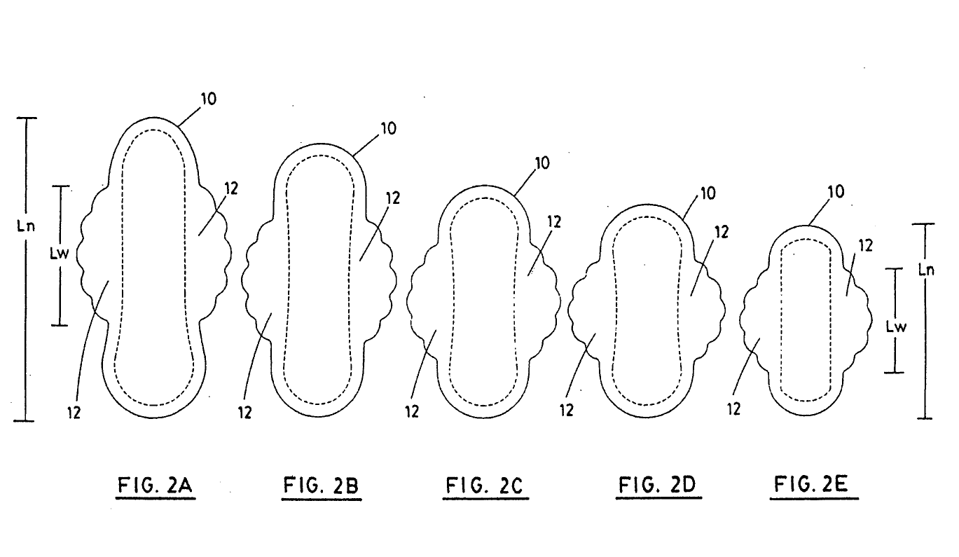

Referring to FIGS. 2A to 2E, there is shown another product line according to a different embodiment of the invention. In this case, the wings have a profile reminiscent of the shape of a flower. As may be observed, the length of the wings again varies proportionally with the length of the whole napkin. It will be noticed that some of the napkins themselves may have a different contour (such as the one in FIG. 2A when compared to FIGS. 2B to 2E) without departing from the scope of the invention.

In accordance with another aspect of the invention, the line of hygiene products comprises different packs, each containing a plurality of sanitary napkins of a same length. In other words, the line may comprise a first pack containing a plurality of the above-described sanitary napkins tailored to meet a light menstrual need, a second pack containing a plurality of the above-described sanitary napkins tailored to meet a medium menstrual flow need, and a third pack containing a plurality of the above-described sanitary napkins tailored to meet a heavy menstrual flow need. The line may preferably further comprise a fourth pack containing a plurality of the above-described sanitary napkins tailored to meet a super heavy menstrual flow need.

As used herein, the term “pack” refers to any type of packaging commonly used to contain sanitary napkins, as for example cardboard boxes or plastic bags.

Alternatively, the sanitary napkins of the line may be sold in a single multi-pack containing sanitary napkins tailored to meet different menstrual flow needs. As for example, the line may comprise a multi-pack containing a plurality of sanitary napkins tailored to meet a light menstrual flow need; a plurality of sanitary napkins tailored to meet a medium menstrual flow need; a plurality of sanitary napkins tailored to meet a heavy menstrual flow need, and also it may comprise a plurality of sanitary napkins tailored to meet a super heavy menstrual flow need.

Of course, numerous modifications could be made to the embodiments above without departing from the scope of the invention as defined in the appended claims.

1. A line of feminine hygiene products comprising a plurality of sanitary napkins of different lengths (Ln) tailored to meet different menstrual flow needs, each sanitary napkin being provided with a pair of lateral wings of a given length (Lw), wherein the Lw/Ln ratio is substantially constant for all the sanitary napkins of the line.

2. A line of feminine hygiene products according to claim 1, wherein the ratio is comprised between 0.32 and 0.34.

3. A line of hygiene products according to claim 1, wherein one sanitary napkin of said plurality of napkins has a first length tailored to meet a light menstrual flow need.

4. A line of hygiene products according to claim 3, wherein said first length is about 220 mm, and the length of the lateral wings associated with said first length is 70 mm.

5. A line of hygiene products according to claim 1, wherein one of said plurality of sanitary napkins has a second length tailored to meet a medium menstrual flow need.

6. A line of hygiene products according to claim 5, wherein said second length is about 240 mm, and the length of the lateral wings associated with said second length is 80 mm.

7. A line of hygiene products according to claim 1, wherein one of said plurality of sanitary napkins has a third length tailored to meet a heavy menstrual flow need.

8. A line of hygiene products according to claim 7, wherein said third length is about 280 mm, and the length of the lateral wings associated with said third length is 90 mm.

9. A line of hygiene products according to claim 1, wherein one of said plurality of sanitary napkins has a fourth length tailored to meet a super heavy or night-time menstrual flow need.

10. A line of hygiene products according to claim 9, wherein said fourth length is about 310 mm, and the length of the lateral wings associated with said fourth length is 100 mm.

11. A line of hygiene products according to claim 7, comprising:
a first pack containing a plurality of said sanitary napkin tailored to meet a light menstrual flow need;
a second pack containing a plurality of said sanitary napkin tailored to meet a medium menstrual flow need; and
a third pack containing a plurality of said sanitary napkin tailored to meet a heavy menstrual flow need.

12. A line of hygiene products according to claim 9, comprising:
a first pack containing a plurality of said sanitary napkin tailored to meet a light menstrual flow need;
a second pack containing a plurality of said sanitary napkin tailored to meet a medium menstrual flow need;
a third pack containing a plurality of said sanitary napkin tailored to meet a heavy menstrual flow need; and
a fourth pack containing a plurality of said sanitary napkin tailored to meet a super heavy menstrual flow need.

13. A line of hygiene products according to claim 7, comprising:
a multi-pack containing a plurality of said sanitary napkin tailored to meet a light menstrual flow need; a plurality of said sanitary napkin tailored to meet a medium menstrual flow need; and a plurality of said sanitary napkin tailored to meet a heavy menstrual flow need.

14. A line of hygiene products according to claim 9, comprising:
a multi-pack containing a plurality of said sanitary napkin tailored to meet a light menstrual flow need; a plurality of said sanitary napkin tailored to meet a medium menstrual flow need; a plurality of said sanitary napkin tailored to meet a heavy menstrual flow need; and a plurality of said sanitary napkin tailored to meet a super heavy menstrual flow need.

15. A line of feminine hygiene products according claim 1, wherein each of said lateral wings is provided with steps on either side of said wing, each step being located in a corner formed by said lateral wing and a corresponding one of said napkins.

16. A line of feminine hygiene products according to claim 1, wherein the profile of the wings is semi-circular with a wavy periphery in the shape of a flower.
